(19) United States
(12) Patent Application Publication
(10) Pub. No.: US 2026/0152561 A1
(43) Pub. Date: Jun. 4, 2026

(54) DUAL-TARGETING CHIMERIC ANTIGEN RECEPTOR, AND ITS ENCODING GENE, RECOMBINANT EXPRESSION VECTOR, NATURAL KILLER CELL, AND APPLICATION

(71) Applicant: Henan Medical University, Xinxiang (CN)

(72) Inventors: Wuling ZHU, Xinxiang (CN); Changjiang GUO, Xinxiang (CN); Mingfeng LI, Xinxiang (CN); Xinru JIN, Xinxiang (CN); Zhiyuan NIU, Xinxiang (CN); Lingtong ZHI, Xinxiang (CN)

(21) Appl. No.: 19/529,076

(22) Filed: Feb. 3, 2026

Related U.S. Application Data
(63) Continuation of application No. PCT/CN2025/099847, filed on Jun. 9, 2025.

(30) Foreign Application Priority Data
Nov. 27, 2024 (CN) 202411714484.6

(51) Int. Cl. C07K 16/28; A61K 40/15; A61K 40/31; A61K 40/42; A61P 35/00; C07K 14/705; C07K 14/725; C12N 5/0783

(52) U.S. Cl. CPC C07K 16/2818; A61K 40/15; A61K 40/31; A61K 40/4202; A61K 40/421; A61P 35/00; C07K 14/7051; C07K 14/70521; C07K 14/70578; C07K 16/2851; C12N 5/0646; C07K 2319/02; C07K 2319/03; C12N 2510/00

(57) ABSTRACT

A dual-targeting chimeric antigen receptor (CAR) and its encoding gene, a recombinant expression vector, natural killer cells, and applications thereof are provided, relating to the field of tumor immunotherapy. The dual-targeting CAR includes a dual-CAR molecular encoding sequence sequentially connected encoding sequences of: an extracellular antigen-binding region, a transmembrane region, and an intracellular signal transduction region. PD1 molecule extracellular domain and NKG2D molecule extracellular domain serve as a first recognition target and a second recognition target respectively. When the PD1 molecule extracellular domain recognizes PD-L1 molecules on tumor cells, the negative regulatory signal is transmitted downward, triggering activation of the DAP10 activating receptor pathway and converting it into a positive activation signal. Simultaneously, when the NKG2D molecule extracellular domain recognizes NKG2DL molecules on tumor cells, NK cell activation is induced. Under the synergistic action of costimulatory molecules 4-1BB and CD3ζ, the anti-tumor therapeutic efficacy of NK cells is significantly enhanced.

### TECHNICAL FIELD

The disclosure relates to the field of tumor immunotherapy technologies, and more particularly to a dual-targeting chimeric antigen receptor, and its encoding gene, recombinant expression vector, natural killer (NK) cell, and application.

### STATEMENT REGARDING SEQUENCE LISTING

The sequence listing associated with this application is provided in text format in lieu of a paper copy and is hereby incorporated by reference into the specification. The name of the XML file containing the sequence listing is 25016MYZ-USP1-SL.xml. The XML file is 43,229 bytes; is created on Jan. 15, 2026; and is being submitted electronically via patent center.

### BACKGROUND

Tumor immunotherapy can effectively overcome the mechanism of tumor immune escape, thus reawakening immune cells to clear tumor cells. Because of its small side effects and obvious therapeutic effect, it is gradually becoming the development direction of tumor treatment in the future, and it is called the fourth largest tumor treatment technology after surgery, radiotherapy and chemotherapy.

Chimeric antigen receptor, abbreviated as CAR, has driven rapid advancements in tumor immunotherapy as its technology continues to evolve. This progress not only accelerates the development of novel therapeutic strategies but also offers renewed hope for cancer patients. The design and application of CAR structures have introduced innovative and transformative methods to cancer treatment. T cells are the first immune cells modified by CAR to specifically target tumor cells, and clinical results show that CAR-T cells have great advantages in the treatment of hematological malignancies. However, with the development of CAR-T technology, it is gradually found that its cell efficacy is limited by the heterogeneity of tumor antigens, and the problem of tumor recurrence often occurs. Therefore, overcoming postoperative recurrence has been a major direction and focus we have been exploring, leading to a gradual transition toward CAR-NK cell therapy in recent years.

In recent years, with people paying more and more attention to the unique characteristics and specific cytotoxicity of natural killer cells, natural killer is abbreviated as NK, and people are increasingly interested in developing CAR-NK cells for cancer treatment. Compared with the CAR-T cells, the CAR-NK cells can provide some significant advantages as follows. (1) The CAR-NK cells have enhanced safety, such as reduced risks of cytokine release syndrome (CRS), neurotoxicity, and graft-versus-host disease (GVHD) in allogeneic environment. (2) The CAR-NK cells have various mechanisms to activate cytotoxic activity. (3) The CAR-NK cells have high feasibility of “off-the-shelf” manufacturing. CAR-NK cells can be transformed to target a variety of antigens, enhance in vivo proliferation and persistence, increase infiltration into solid tumors, overcome drug-resistant tumor microenvironment, and finally achieve effective anti-tumor response. The NK cells seem to be an attractive vector for CAR expression, because they can be obtained from a variety of sources and can be injected safely, regardless of the matching between donors and patients, which greatly reduces the cost of treatment. The CAR-NK cells are known to be effective in hematological malignancies, but more and more preclinical findings show that they are still active in non-hematological tumors.

Although the advantages of the CAR-NK cell therapy over CAR-T cell therapy are obvious, there are still cases where patients are prone to develop drug resistance or tumor recurrence due to antigen escape. Therefore, it is an urgent problem to study a CAR targeting two antigens to prevent antigen escape.

### SUMMARY

Immune checkpoint refers to a kind of immunosuppressive molecules, which regulate the activity of immune cells through a series of ways of co-inhibition or co-stimulation signals, prevent excessive immune activation and ensure self-tolerance, thus avoiding normal tissue damage and destruction. Programmed cell death protein 1 (PD1) is one of the main inhibitory molecules. Negative immunoregulatory mechanism mediated by PD1/programmed death-ligand 1 (PD-L1) can inhibit the activation of major histocompatibility complex-T cell receptor (MHC-TCR) signaling pathway, and control the proliferation of T cells and NK cells, cytokine secretion or response to similar antigens. PD1 is mainly expressed on the surface of activated immune cells. The expression of its ligand PD-L1 is limited in normal tissues, but it is significantly increased in most solid tumor cells and immunosuppressed cells. Tumors utilize the negative immunoregulatory mechanism of PD1/PD-L1 to escape the anti-tumor immune response. Therefore, blocking this immunosuppressive signaling pathway has become an important path for immunotherapy of solid tumors. Natural killer group 2D ligands (NKG2DLs) are diverse and exist in many kinds of tumor cells. It has been reported that high expression of MHC class I polypeptide-related sequence A/B (MICA/B) can often be detected in liver cancer, ovarian cancer, pancreatic cancer, colon cancer, breast cancer, etc., but NKG2DLs is rarely expressed in healthy cells. Therefore, in the process of cell carcinogenesis, NK cells, which play an immune monitoring role in the immune system, can well recognize and bind with these ligands through their own natural killer group 2, member D (NKG2D) receptors, thus triggering the activation effect of NK cells and effectively eliminating various tumor cells.

In the disclosure, the PD1-DAP10-41BB-P2A-NKG2D-IgG4-CD28-41BB-CD3ζ fusion receptor is synthesized by using a whole gene synthesis technology, that is, only an extracellular domain of PD1 molecule is reserved as a first recognition target, and the intracellular inhibitory signaling domain of PD1 receptor is replaced by the transmembrane and intracellular domains of DNAX-activating protein of 10 kDa (DAP10) and an intracellular domain of costimulatory molecule tumor necrosis factor receptor superfamily member 9 (4-1BB, also referred to as CD137). This chimeric molecular module can play the role of “switch” in signal conversion, thus reversing the negative immunoregulatory mechanism of PD1/PD-L1. At the same time, the extracellular domain of NKG2D molecule is selected as a second recognition target, and the transmembrane and intracellular domains of CD28, the intracellular domains 4-1BB and CD3 zeta chain (CD3ζ) are used as costimulatory molecules. When the extracellular signaling domain of PD1 recognizes the PD-L1 molecule on tumor cells, the negative regulatory signal is transmitted downwards, which triggers the activation of DAP10 activated receptor pathway and become a positive activation signal. At the same time, when the extracellular domain of NKG2D molecule recognizes NKG2DLs molecule on tumor cells, it can also activate NK cells, and under the synergistic effect of costimulatory molecules 4-1BB and CD3ζ, the anti-tumor effect of NK cells is greatly enhanced.

Specifically, a dual-targeting chimeric antigen receptor includes the amino acid sequence as shown in SEQ ID NO: 3. The dual-targeting chimeric antigen receptor is divided into two sections, including a first receptor including a extracellular domain PD1, extracellular transmembrane and intracellular domains DAP10 and a costimulatory molecule CD137, and a second receptor including an extracellular domain NKG2D, a hinge region structure IgG4, transmembrane and intracellular domains cluster of differentiation 28 (CD28), an intracellular domain CD137 and an intracellular domain CD3ζ, which can be quickly combined in cells to form a dual-targeting receptor. Using this chimeric molecular module, on the one hand, NKG2D is combined with MICA/MICB on the tumor surface, and PD1 is combined with PD-L1 of the tumor cell, so that the function of targeting the tumor cell is fully exerted; and on the other hand, the activation effect of NK92 cells is enhanced through the combined action of NKG2D and DAP10.

In an embodiment, the coding gene of the dual-targeting chimeric antigen receptor is initiated by a cytomegalovirus (CMV) promoter, and the first receptor and the second receptor are connected by a Porcine teschovirus-1 2A (P2A) peptide, so that the phenomenon of uneven target protein expression of the double promoters is avoided. The nucleotide sequence accords with codon preference in human body and can be better expressed in human body.

In an embodiment, in the targets PD1 and NKG2D of the two receptors, a signal peptide region (also referred to as extracellular signal peptide structure) has a signal peptide sequence related to protein secretion, which can be selected from type I transmembrane protein signal peptides such as CD8α, PD1, DAP10, DNAM-1, CD137 (also referred to as 4-1BB). Specifically, it is CD8αsignal peptide. The amino acid sequence of the CD8αsignal peptide is shown in SEQ ID NO: 1, and the nucleotide sequence encoding the amino acid sequence is shown in SEQ ID NO: 14.

In an embodiment, in the chimeric receptor targeting the NKG2D ligand, the hinge region is specifically a haplotype IgG4 hinge region (i.e., IgG4 molecule). The amino acid sequence of the IgG4 molecule is shown in SEQ ID NO: 8, and the nucleotide sequence encoding the amino acid sequence is shown in SEQ ID NO: 21.

In an embodiment, in the chimeric receptor targeting NKG2D ligand, a Flag tag is further connected between the signal peptide region and the binding domain of NKG2D ligand. The amino acid sequence of the Flag tag is shown in SEQ ID NO: 6, and the nucleotide sequence encoding the amino acid sequence is shown in SEQ ID NO: 19. In the chimeric receptor targeting PD-L1 ligand, a MYC tag is further connected between the signal peptide region and the binding domain of PD-L1 ligand. The amino acid sequence of the MYC tag is shown in SEQ ID NO: 2, and the nucleotide sequence encoding the amino acid sequence is shown in SEQ ID NO: 15.

The disclosure relates to a recombinant expression vector containing a coding gene of a dual-targeting chimeric antigen receptor, and the amino acid sequence of the dual-targeting chimeric antigen receptor is shown in SEQ ID NO: 13.

A preparation method of the recombinant expression vector including the following steps:
1) synthesizing the coding gene of the dual-targeting chimeric antigen receptor; and
2) connecting the coding gene of the dual-targeting chimeric antigen receptor to a lentivirus expression vector, so as to obtain the recombinant expression vector.

The disclosure relates to an anti-tumor NK cell with high cytotoxicity, which is an NK cell modified by a dual-targeting chimeric antigen receptor, and the amino acid sequence of the dual-targeting chimeric antigen receptor is shown in SEQ ID NO: 13

The anti-tumor NK cell of the disclosure has extremely strong cytotoxicity. Compare with the targeting effect of CAR-NK on a certain antigen, PN-CAR-NK92 cells recognize tumor cells through NKG2D/NKG2DL and PD1/PD-L1 pathways, and because most tumors express MICA, MICB and PD-L1, the targeting effect of the PN-CAR-NK92 on the tumor cells has a broad spectrum and is not limited to one or multiple specific antigen targets.

A preparation method of the anti-tumor NK cells includes the following steps:
1) synthesizing the coding gene of the dual-targeting chimeric antigen receptor;
2) connecting the coding gene of the dual-targeting chimeric antigen receptor to a lentivirus expression vector to obtain a recombinant lentivirus expression vector;
3) packaging the recombinant lentivirus expression vector to express a virus carrying a target plasmid; and
4) infecting NK92 cells with the virus carrying the target plasmid, thus obtaining the anti-tumor NK cells.

The anti-tumor NK cells of the disclosure can be used for allogeneic reinfusion, have small damage to normal cells, cannot cause excessive immune response of an organism, can realize accurate identification and specific killing of solid tumor cells, and have stronger cytotoxicity and better treatment effect on tumors.

### BRIEF DESCRIPTION OF DRAWINGS

FIG. 1 illustrates a schematic structural diagram of a recombinant lentivirus expression vector CMV-PD1-DAP10-41BB-P2A-NKG2D-IgG4-CD28-41BB-CD3ζ-pCDH containing a coding gene of a dual-targeting chimeric antigen receptor.

FIG. 2 illustrates a schematic diagram of messenger ribonucleic acid (mRNA) level expression of PD1 and NKG2D in molecular module PN-CAR-NK92 cells after lentivirus transfection according to an embodiment 3 of the disclosure.

FIG. 3 illustrates a schematic flow cytometry detection diagram of the PD1 and the NKG2D of the molecular module PN-CAR-NK92 cells after lentivirus transfection according to an embodiment 4 of the disclosure.

FIG. 4 illustrates a schematic flow cytometry detection diagram of PD-L1 and MICA/MICB of EMT6-PD-L1 and MGC-803 tumor cells according to an embodiment 5 of the disclosure.

FIG. 5 illustrates killing efficiency of NK92, single-module PD1-CAR-NK92, NG2D-CAR-NK92 and dual-module PN-CAR-NK92 cells on the EMT6-PD-L1 and MGC-803 tumor cells according to an embodiment 6 of the disclosure.

FIG. 6 illustrates a schematic flow cytometry diagram of PD-L1 and MICA/MICB of KATO-III and U2OS tumor cells according to an embodiment 5 of the disclosure.

FIG. 7 illustrates killing efficiency of NK92 and PN-CAR-NK92 cells on the KATO-III and U2OS tumor cells according to an embodiment 6 of the disclosure.

FIG. 8 illustrates a schematic diagram of a rational design of the PN-CAR-NK92 cells and anti-tumor effect of modified NK cells.

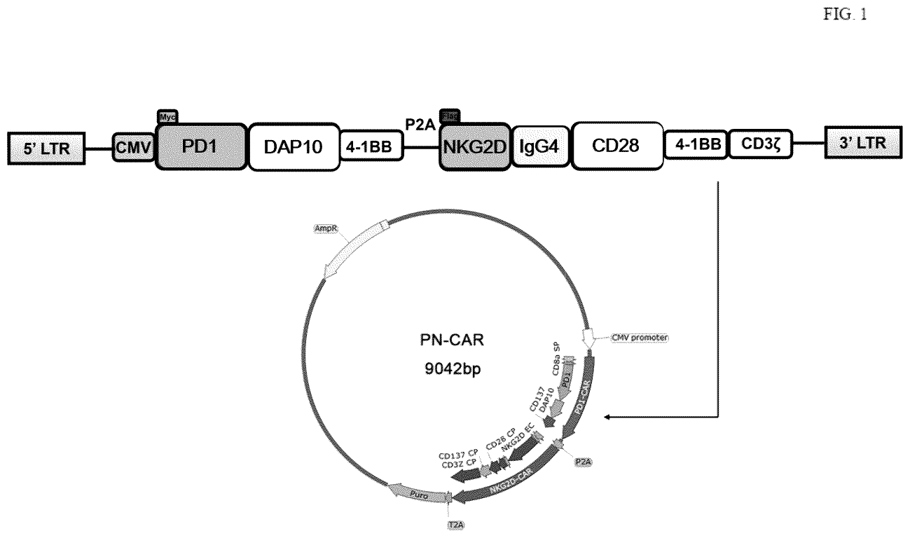

### DETAILED DESCRIPTION OF EMBODIMENTS

Specific embodiments of the disclosure are described in detail below. However, it should be understood that the scope of protection of the disclosure is not limited by these specific embodiments. Based on the embodiments in the disclosure, all other embodiments obtained by those skilled in the art without making creative labor belong to the scope of protection of the disclosure. Experimental methods mentioned in the various embodiments of the disclosure are all conventional methods unless otherwise specified.

In the disclosure, an extracellular recognition binding region recognizes any tumor-specific or tumor-associated antigen, including PD1 ligands (PD-L1, PD-L2) and NKG2D ligands (MICA, MICB, ULBP1, ULBP2, ULBP3, ULBP4, ULBP5, ULBP6, etc.).

### Embodiment 1

A dual-targeting chimeric antigen receptor consists of a PD1-DAP10-CD137 receptor and an NKG2D-IgG4-CD28-CD137-CD32 receptor.

The PD1-DAP10-CD137 receptor consists of a CD8αsignal peptide, a Myc tag, the PD1 extracellular domain, the DAP10 transmembrane and intracellular domains, and the CD137 intracellular domain.

The amino acid sequence of the CD8αsignal peptide is:

MALPVTALLLPLALLLHAARP, designated as SEQ ID NO: 1.

The amino acid sequence of the Myc tag is:

EQKLISEEDL, designated as SEQ ID NO: 2.

The amino acid sequence of the PD1 extracellular domain is:

FLDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLV,
designated as SEQ ID NO: 3.

The amino acid sequence of the DAP10 transmembrane and intracellular domain is:

QTTPGERSSLPAFYPGTSGSCSGCGSLSLPLLAGLVAADAVASLLIVGAV
FLCARPRRSPAQEDGKVYINMPGRG, designated as SEQ ID
NO: 4.

The amino acid sequence of the CD137 intracellular domain is:

KRGRKKLLYIFKQPFMRPVQTTQEEDGCSCRFPEEEEGGCEL,
designated as SEQ ID NO: 5.

The NKG2D-IgG4-CD28-CD137-CD35 receptor consists of a CD8αsignal peptide, a FLAG tag, the NKG2D ligand-binding domain, an IgG4 haplotype hinge region, the CD28 transmembrane and intracellular domain, the CD137 intracellular domain, and the CD35 intracellular domain.

The amino acid sequence of the FLAG tag is:

DYKDDDDK, designated as SEQ ID NO: 6.

The amino acid sequence of the NKG2D ligand-binding domain is:

LFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTI
IEMQKGDCALYASSFKGYIENCSTPNTYICMQRTV, designated as
SEQ ID NO: 7.

The amino acid sequence of the IgG4 haplotype hinge region:

ESKYGPPAPPAP, designated as SEQ ID NO: 8.

The amino acid sequence of the CD28 transmembrane and intracellular domain is:

FWVLVVVGGVLACYSLLVTVAFIIFWVRSKRSRLLHSDYMNMTPRRPGPT
RKHYQPYAPPRDFAAYRS, designated as SEQ ID NO: 9.

The amino acid sequence of the CD137 intracellular domain is:

KRGRKKLLYIFKQPFMRPVQTTQEEDGCSCRFPEEEEGGCEL,
designated as SEQ ID NO: 10.

The amino acid sequence of the CD3 intracellular domain is:

RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR, designated as SEQ ID NO: 11.

The PD1-DAP10-CD137 receptor is prepared according to steps as follows.

A first receptor is prepared by synthesizing a nucleotide fragment containing the PD1 extracellular domain (1-147 aa), a nucleotide fragment containing the DAP10 extracellular, transmembrane, and intracellular domains (19-93 aa), and a nucleotide fragment containing the CD137 (also known as 4-1BB) intracellular domain (214-255 aa) using a whole gene synthesis method.

The NKG2D-IgG4-CD28-CD137-CD35 receptor is prepared according to steps as follows.

A second receptor is prepared by synthesizing a nucleotide fragment containing the NKG2D extracellular domain sequence, sequentially linked to the IgG4 hinge region sequence, the CD28 transmembrane domain sequence (153-220 aa), the CD137 intracellular domain sequence, and the CD3ζ intracellular domain sequence, using a whole gene synthesis method.

The dual-targeting chimeric antigen receptor is prepared according to the following steps.

The nucleotide fragment of the first receptor and the nucleotide fragment of the second receptor are concatenated via a self-cleaving peptide P2A linker using a whole gene synthesis method, resulting in the dual-targeting chimeric antigen receptor, namely the PD1-DAP10-41BB-P2A-NKG2D-IgG4-CD28-41BB-CD3ζ fragment.

The nucleotide sequence encoding the dual-targeting chimeric antigen receptor is:

designated as SEQ ID NO: 12
ATGGCGCTTCCGGTGACTGCTCTACTTCTACCGTTGGCTCTGCTGCTCCACGCCGC
TCGCCCTGAACAGAAGCTCATCTCGGAGGAAGACCTGTTCCTGGACAGCCCCGAC
CGCCCGTGGAATCCACCCACCTTTTCACCGGCCCTGCTGGTGGTTACAGAGGGCG
ACAACGCCACGTTCACCTGCTCCTTCTCCAACACGTCGGAGAGTTTCGTCCTCAAC
TGGTACAGGATGTCTCCCAGCAACCAGACCGACAAACTTGCTGCGTTCCCCGAGG
ATCGCAGCCAGCCGGGCCAGGATTGCCGATTCAGAGTCACCCAGCTGCCCAACG
GTCGGGACTTTCATATGTCCGTGGTCCGCGCCCGCCGCAACGACTCTGGCACCTA
CCTGTGCGGGGCCATCTCTTTGGCTCCCAAGGCCCAAATCAAGGAGAGCCTGCGC
GCCGAGCTCCGCGTAACCGAGCGGCGTGCAGAGGTGCCAACCGCTCACCCCTCTC
CTAGCCCTAGGCCTGCCGGACAGTTCCAGACCCTGGTGCAGACTACTCCCGGTGA
GCGCAGTTCCCTGCCTGCGTTTTATCCGGGGACCAGCGGCTCCTGTTCTGGATGTG
GTTCGCTGTCCTTGCCCCTGCTGGCCGGCTTGGTGGCGGCGGACGCCGTGGCTTCC
CTGCTGATTGTCGGTGCCGTGTTCCTGTGCGCCCGGCCTCGCCGTTCCCCAGCGCA
GGAGGACGGCAAAGTGTACATTAATATGCCCGGCCGCGGCAAGCGCGGGCGAAA
GAAGCTGCTGTACATCTTCAAGCAGCCATTTATGCGTCCTGTGCAGACGACCCAG
GAGGAAGATGGCTGTTCATGCCGCTTCCCGGAGGAGGAGGAGGGCGGCTGCGAG
TTGgaattcgaatttaaaggatcCcgcggccgcGCTAGCGGAAGCGGAGCTACTAACTTCAGCCTG
CTGAAGCAGGCTGGAGACGTGGAGGAGAACCCTGGACCTATGGCCTTACCAGTG
ACCGCCTTGCTCCTGCCGCTGGCCTTGCTGCTCCACGCCGCCAGGCCGGACTACA
AAGACGATGACGACAAGTTATTCAACCAAGAAGTTCAAATTCCCTTGACCGAAA
GTTACTGTGGCCCATGTCCTAAAAACTGGATATGTTACAAAAATAACTGCTACCA
ATTTTTTGATGAGAGTAAAAACTGGTATGAGAGCCAGGCTTCTTGTATGTCTCAA
AATGCCAGCCTTCTGAAAGTATACAGCAAAGAGGACCAGGATTTACTTAAACTGG
TGAAGTCATATCATTGGATGGGACTAGTACACATTCCAACAAATGGATCTTGGCA
GTGGGAAGATGGCTCCATTCTCTCACCCAACCTACTAACAATAATTGAAATGCAG
AAGGGAGACTGTGCACTCTATGCCTCGAGCTTTAAAGGCTATATAGAAAACTGTT
CAACTCCAAATACGTACATCTGCATGCAAAGGACTGTGGAATCTAAATATGGGCC
TCCAGCACCACCCGCGCCTTTTTGGGTGCTGGTGGTGGTTGGTGGAGTCCTGGCTT
GCTATAGCTTGCTAGTAACAGTGGCCTTTATTATTTTCTGGGTGAGGAGTAAGAG
GAGCAGGCTCCTGCACAGTGACTACATGAACATGACTCCCCGCCGCCCCGGGCCC
ACCCGCAAGCATTACCAGCCCTATGCCCCACCACGCGACTTCGCAGCCTATCGCT
CCAAACGGGGCAGAAAGAAACTCCTGTATATATTCAAACAACCATTTATGAGACC
AGTACAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCCCCGC
GTACCAGCAGGGCCAGAACCAGCTCTATAACGAGCTCAATCTAGGACGAAGAGA
GGAGTACGATGTTTTGGACAAGAGACGTGGCCGGGACCCTGAGATGGGGGGAAA
GCCGCAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTA
CGACGCCCTTCACATGCAGGCCCTGCCCCCTCGA,.

The amino acid sequence of the dual-targeting chimeric antigen receptor is:

designated as SEQ ID NO: 13
MALPVTALLLPLALLLHAARPDYKDDDDKLFNQEVQIPLTESYCGPCPKNWICYKNN
CYQFFDESKNWYESQASCMSQNASLLKVYSKEDQDLLKLVKSYHWMGLVHIPTNGS
WQWEDGSILSPNLLTIIEMQKGDCALYASSFKGYIENCSTPNTYICMQRTVESKYGPP
APPAPFWVLVVVGGVLACYSLLVTVAFIIFWVRSKRSRLLHSDYMNMTPRRPGPTRK
HYQPYAPPRDFAAYRSKRGRKKLLYIFKQPFMRPVQTTQEEDGCSCRFPEEEEGGCE
LRVKFSRSADAPAYQQGQNQLYNELNLGRREEYDVLDKRRGRDPEMGGKPQRRKN
PQEGLYNELQKDKMAEAYSEIGMKGERRRGKGHDGLYQGLSTATKDTYDALHMQA
LPPR,.

The nucleotide sequence of the CD8α-C1 signal peptide is:

designated as SEQ ID NO: 14
ATGGCGCTTCCGGTGACTGCTCTACTTCTACCGTTGGCTCTGCTGCTCC
ACGCCGCTCGCCCT,.

The nucleotide sequence of the Myc tag is:

designated as SEQ ID NO: 15
GAACAGAAGCTCATCTCGGAGGAAGACCTG,.

The nucleotide sequence of the PD1 extracellular domain is:

designated as SEQ ID NO: 16
TTCCTGGACAGCCCCGACCGCCCGTGGAATCCACCCACCTTTTCACCGG
CCCTGCTGGTGGTTACAGAGGGCGACAACGCCACGTTCACCTGCTCCTT
CTCCAACACGTCGGAGAGTTTCGTCCTCAACTGGTACAGGATGTCTCCC
AGCAACCAGACCGACAAACTTGCTGCGTTCCCCGAGGATCGCAGCCAGC
CGGGCCAGGATTGCCGATTCAGAGTCACCCAGCTGCCCAACGGTCGGGA
CTTTCATATGTCCGTGGTCCGCGCCCGCCGCAACGACTCTGGCACCTAC
CTGTGCGGGGCCATCTCTTTGGCTCCCAAGGCCCAAATCAAGGAGAGCC
TGCGCGCCGAGCTCCGCGTAACCGAGCGGCGTGCAGAGGTGCCAACCGC
TCACCCCTCTCCTAGCCCTAGGCCTGCCGGACAGTTCCAGACCCTGGT
G,.

The nucleotide sequence of the DAP10 transmembrane and intracellular domains is:

designated as SEQ ID NO: 17
CAGACTACTCCCGGTGAGCGCAGTTCCCTGCCTGCGTTTTATCCGGGGA
CCAGCGGCTCCTGTTCTGGATGTGGTTCGCTGTCCTTGCCCCTGCTGGC
CGGCTTGGTGGCGGCGGACGCCGTGGCTTCCCTGCTGATTGTCGGTGCC
GTGTTCCTGTGCGCCCGGCCTCGCCGTTCCCCAGCGCAGGAGGACGGCA
AAGTGTACATTAATATGCCCGGCCGCGGC,.

The nucleotide sequence of the CD137-C1 intracellular domain is:

designated as SEQ ID NO: 18
AAGCGCGGGCGAAAGAAGCTGCTGTACATCTTCAAGCAGCCATTTATGC
GTCCTGTGCAGACGACCCAGGAGGAAGATGGCTGTTCATGCCGCTTCCC
GGAGGAGGAGGAGGGCGGCTGCGAGTTG,.

The nucleotide sequence of the CD8α-C2 signal peptide is:

designated as SEQ ID NO: 18
ATGGCCTTACCAGTGACCGCCTTGCTCCTGCCGCTGGCCTTGCTGCTCC
ACGCCGCCAGGCCG,.

The nucleotide sequence of the FLAG tag is:

designated as SEQ ID NO: 19
GACTACAAAGACGATGACGACAAG,.

The nucleotide sequence of the NKG2D ligand-binding domain is:

designated as SEQ ID NO: 20
TTATTCAACCAAGAAGTTCAAATTCCCTTGACCGAAAGTTACTGTGGCC
CATGTCCTAAAAACTGGATATGTTACAAAAATAACTGCTACCAATTTTT
TGATGAGAGTAAAAACTGGTATGAGAGCCAGGCTTCTTGTATGTCTCAA
AATGCCAGCCTTCTGAAAGTATACAGCAAAGAGGACCAGGATTTACTTA
AACTGGTGAAGTCATATCATTGGATGGGACTAGTACACATTCCAACAAA
TGGATCTTGGCAGTGGGAAGATGGCTCCATTCTCTCACCCAACCTACTA
ACAATAATTGAAATGCAGAAGGGAGACTGTGCACTCTATGCCTCGAGCT
TTAAAGGCTATATAGAAAACTGTTCAACTCCAAATACGTACATCTGCAT
GCAAAGGACTGTG,.

The nucleotide sequence of the IgG4 haplotype hinge region is:

designated as SEQ ID NO: 21
GAATCTAAATATGGGCCTCCAGCACCACCCGCGCCT,.

The nucleotide sequence of the CD28 transmembrane and intracellular domains is:

designated as SEQ ID NO: 22
TTTTGGGTGCTGGTGGTGGTTGGTGGAGTCCTGGCTTGCTATAGCTTGC
TAGTAACAGTGGCCTTTATTATTTTCTGGGTGAGGAGTAAGAGGAGCAG
GCTCCTGCACAGTGACTACATGAACATGACTCCCCGCCGCCCCGGGCCC
ACCCGCAAGCATTACCAGCCCTATGCCCCACCACGCGACTTCGCAGCCT
ATCGCTCC,.

The nucleotide sequence of the CD137-C2 intracellular domain is:

designated as SEQ ID NO: 23
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG,.

The nucleotide

designated as SEQ ID NO: 24
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
CAGAGAAGGAAGAACCCTCAGGAAGGCCTGTACAATGAACTGCAGAAAG
ATAAGATGGCGGAGGCCTACAGTGAGATTGGGATGAAAGGCGAGCGCCG
GAGGGGCAAGGGGCACGATGGCCTTTACCAGGGTCTCAGTACAGCCACC
AAGGACACCTACGACGCCCTTCACATGCAGGCCCTGCCCCCTCGA,.

sequence of the CD35 intracellular domain is:

### Embodiment 2

A preparation method of a recombinant expression vector encoding a dual-targeting chimeric antigen receptor includes steps as follows.

A first receptor and a second receptor are synthesized by a whole gene synthesis method, the first receptor consists of nucleotide fragments encoding the PD1 extracellular domain (1-150 aa), the DAP10 extracellular, transmembrane, and intracellular domains (19-93 aa), and the CD137 (also referred to as 4-1BB) intracellular domain (214-255 aa). The second receptor is synthesized by sequentially linking the NKG2D extracellular domain sequence to the IgG4 hinge region sequence (monomer mutant: ESKYGPPAPPAP), the CD28 transmembrane domain sequence (153-179 aa), the CD137 intracellular domain, and the CD35 intracellular domain. The two receptors are linked via a self-cleaving peptide P2A. The nucleotide fragments of the first receptor and the nucleotide fragments of the second receptor are concatenated to obtain the PD1-DAP10-41BB-P2A-NKG2D-IgG4-CD28-41BB-CD35 fragment.

The coding gene of the dual-targeting chimeric antigen receptor is synthesized, with its nucleotide sequence shown as SEQ ID NO: 12.

The coding gene of the dual-targeting chimeric antigen receptor is ligated into a lentivirus expression vector, including steps as follows.

The PD1-DAP10-41BB-P2A-NKG2D-IgG4-CD28-41BB-CD35 fragment and the lentivirus vector (pCDH-CMV-MCS-P2A-copGFP-T2A-Puro) are double-digested with restriction enzymes (Xbal and Xhol). The dual-targeting chimeric antigen receptor fragment and the lentivirus vector are ligated using T4 DNA ligase. The complete lentivirus plasmid is sequenced. The sequencing results match the expected sequences of all fragments, yielding the recombinant expression vector.

### Embodiment 3

A preparation method of anti-tumor NK cells with high cytotoxicity in this embodiment includes steps as follows.

### (I) Lentivirus Packaging

### 1. Extraction of Lentivirus Packaging Plasmid and Target Plasmid

### 1.1 Plasmid Transformation

Stb13 competent cells and Escherichia coli (E. coli) TOP10 competent cells are used. 20 nanograms (ng) of the target plasmid is added to the stb13 competent cells. 1 microgram (μg) of the lentivirus packaging plasmid pSPAX2 and 1 μg of the lentivirus packaging plasmid PMD2.G are added to the E. coli TOP10 competent cells respectively and then mixed gently to obtain mixtures. The mixtures are incubated on ice for 30 minutes (min), immediately transferred to a 42° C. water bath for heat shock for 90 seconds(s), and then placed on ice for 2 min. Subsequently, the cells are incubated in a 37° C. constant-temperature shaker at 220 revolutions per minutes (rpm) for 1 hour (h). A 100 microliters (μL) aliquot is spread onto Luria-Bertani (LB) agar plates and cultured at 37° C. for 13 h.

Stb13 competent cells are purchased from TransGenBiotech (Cat. No. L681212).

The target plasmid is CMV-PD1-DAP10-41BB-P2A-NKG2D-IgG4-CD28-41BB-CD3ζ-pCDH.

The LB agar plates are supplemented with ampicillin (Amp) at a volume ratio of 1:1000 at a concentration of 50 micrograms per milliliter (μg/mL).

### 1.2 Plasmid Extraction

1) Small-scale extraction: single colonies from the aforementioned plates are picked and inoculated into 5 mL of fresh LB broth and incubated in a 37° C. shaker at 220 rpm for 12-14 h. The LB broth is supplemented with Amp at a volume ratio of 1:1000 at a concentration of 50 μg/mL.
2) Large-scale extraction: 200 μL of bacterial culture grown in the step 1) is inoculated into 200 mL of LB broth and shaken at 220 rpm and 37° C. until reaching an optical density at 600 nanometers (OD600)≈1.8. The LB broth contains 200 μL Amp.
3) Plasmids are extracted according to an endotoxin-free plasmid mass extraction kit (OMEGA Bio-Tek), and the concentration and purity of plasmids are measured by a NanoDrop spectrophotometer, and then stored in a refrigerator at −20° C. for subsequent use. Specifically, the endotoxin-free plasmid mass extraction kit is purchased from OMEGA Bio-Tek, USA; Cat. No. D6926-03. The NanoDrop spectrophotometer is purchased from Thermo Fisher Scientific (Shanghai) Instrument Co., Ltd., China, model: NanoDrop2000.

### 2. Lentivirus Packaging Process

When the density of 293 T cells reaches 80%-90%, the cells are cultured in a fresh Dulbecco's modified eagle's (DMEM) high glucose medium containing 6% fetal bovine serum (FBS) for 1 h in an incubator, and the viruses are packaged after 1 h.

Lentiviral packaging is performed using a calcium phosphate kit. The procedure is as follows. The target plasmid CMV-PD1-DAP10-41BB-P2A-NKG2D-IgG4-CD28-41BB-CD33-pCDH is mixed with helper plasmids pSPAX2 and pMD2.G at a 4:3:1 molar ratio to form mixed plasmids. The mixed plasmids are added to a calcium chloride (CaCl₂)) solution provided in the calcium phosphate kit, blown and mixed, and then added to dropwise at a constant rate into a buffered balanced salt (BBS) solution, blown and mixed, and incubated at room temperature for 20 min to obtain the mixed solution. The mixed solution is added to 293 T cells, gently mixed, and cultured in an incubator for 12 h. The supernatant is removed and gently washed with phosphate buffer saline (PBS). 15 mL fresh DMEM medium containing 10% FBS is replaced, and the lentiviruses are collected for 48 h and 72 h, respectively.

The FBS is purchased from Hyclone, Cat. No. SV30265.01B, the DMEM high glucose medium is purchased from Hyclone, Cat. No. WH0021D031, and calcium phosphate kit is purchased from Beyotime Biotechnology (Shanghai, China), Cat. No. C0508.

### 3. Lentivirus Concentration

The collected lentivirus supernatant is centrifuged to remove cell debris, added with polyethylene glycol 8000 (PEG-8000), and incubated at 4° C. for more than 12 hours followed by centrifugation to pellet viral particles. The concentrated viral particles are resuspended in pre-cooled PBS to obtain a concentrated PD1-DAP10-41BB virus solution, which is then aliquoted and stored at −80° C. to avoid repeated freeze-thaw cycles.

### 4. Lentivirus Titer Determination

The lentiviral titer obtained in the embodiment is measured using a quantitative polymerase chain reaction (qPCR) lentivirus titer kit (Cat. No. LV900) from abm Company. The titer is higher than 1×10⁹ Transducing Units, TU/mL.

### (II) Lentiviral Transfected NK92 Cells

NK92 cells are seeded at a density of 3×10⁵ to 5×10⁵ cells per well in a 24-well plate. The concentrated PD1-DAP10-41BB virus solution is added with a multiplicity of infection (MOI) of 50-100. Polybrene with a final concentration of 8 μg/mL is added to each well, mixed, and incubated in a 37° C. incubator for 12-15 h. After incubation, the cells are centrifuged to remove the viral supernatant, and a fresh medium is added to resume cell growth. The resulting lentivirus-infected NK92 cells are designated as PN-CAR-NK92.

The polyamide is purchased from Yeasen Biotechnology Group Co., Ltd., Cat. No. 40804ES76.

(III) mRNA Expression Levels of PD1 and NKG2D by qPCR Detection

PN-CAR-NK92 and NK92 cells infected with lentivirus for 72 h are collected. The mRNA levels of PD1 and NKG2D in PN-CAR-NK92 and NK92 cells are detected by using the following primers:

PD1-F:
(SEQ ID NO: 25)
GTGTCACACAACTGCCCAAC;
PD1-R:
(SEQ ID NO: 26)
CCGCAGGCTCTCTTTGATCT;
NKG2D-F:
(SEQ ID NO: 27)
GAGTGATTTTTCAACACGATGGC;
NKG2D-R:
(SEQ ID NO: 28)
ACAGTAACTTTCGGTCAAGGGAA;
GAPDH-F:
(SEQ ID NO: 29)
GAGGACCTGACCTGCCGTCT;
and
GAPDH-R:
(SEQ ID NO: 30)
GGAGGAGTGGGTGTCGCTGT.

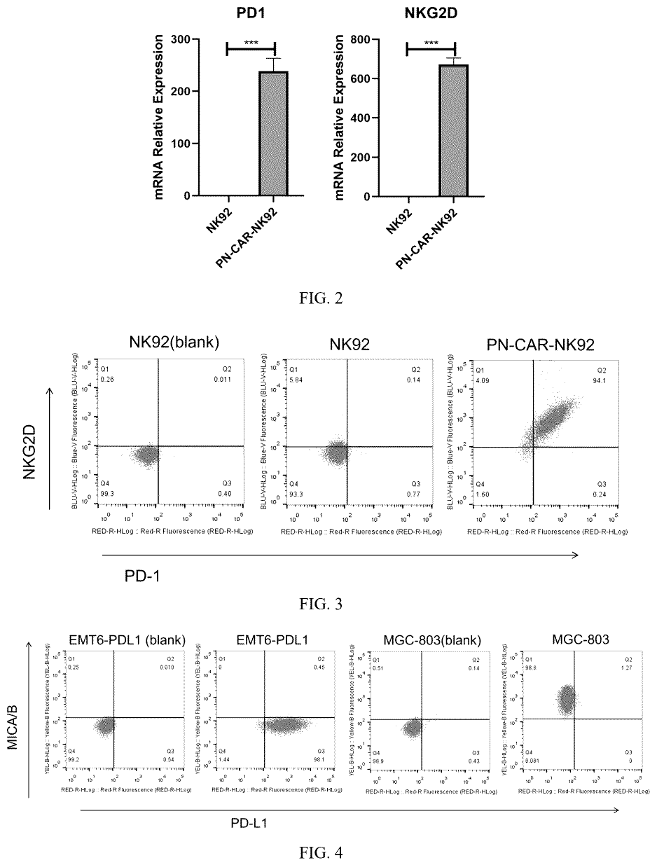

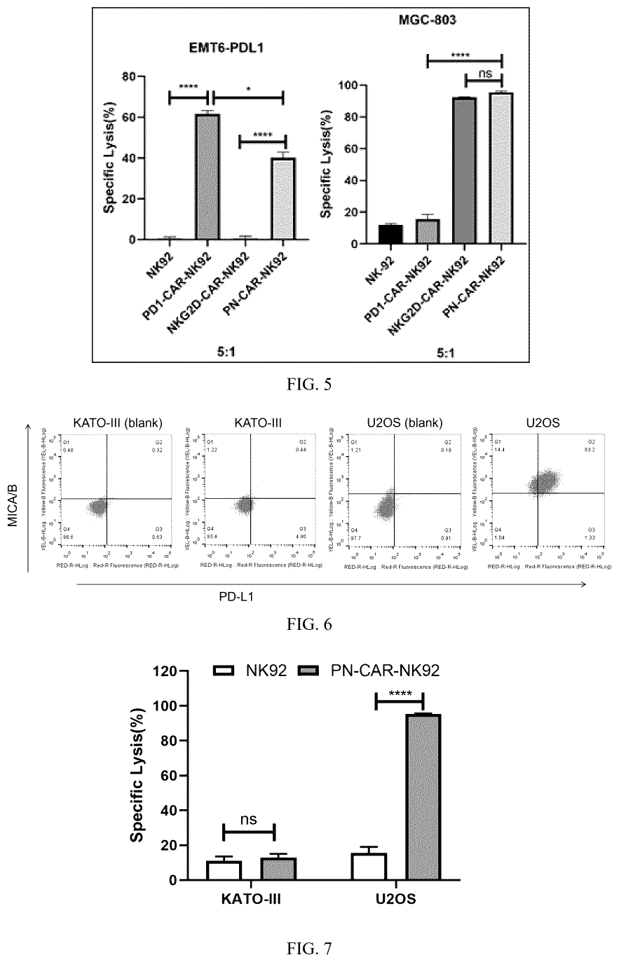

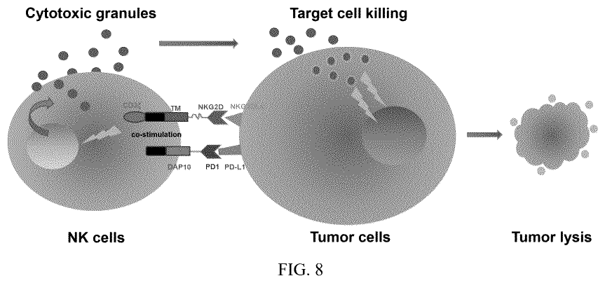

The detection results are shown in FIG. 2, from which it can be seen that the mRNA expression levels of PD1 and NKG2D in the PN-CAR-NK92 cells are much higher than that in the NK92 cells.

### Embodiment 4

### Expressions of the Target Genes PD1 and NKG2D Detected by Flow Cytometry

After 72 h of lentiviral infection, the PN-CAR-NK92 and the NK92 cells are collected and centrifuged at 300×g for 5 min, and 1×10⁶ cells are counted and washed 1-2 times with PBS containing 1% FBS (referred to as a wash buffer hereafter). In a 100 μL volume, allophycocyanin (APC)-conjugated anti-human PD1 antibody and brilliant violet 421™ (BV421)-conjugated anti-human NKG2D antibody are added, incubated on ice in the dark for 30 min, and then washed 2-3 times with the wash buffer. Finally, the cells are resuspended in 400 μL of PBS and analyzed using a flow cytometry.

The APC-conjugated anti-human PD1 antibody is purchased from BD Company, Cat. No. 320822. The flow cytometer used is BD FACSCanto II.

The PD1 detection results are shown in FIG. 3. It can be seen that the expression level of PD-1 in the PN-CAR-NK92 cells is approximately 94.3%, and that of NKG2D is approximately 98.1%. These expression levels are significantly higher compared to those in the NK92 cells.

### Embodiment 5

Detection of PD-L1 expression and MICA/B expression in EMT6-PD-L1, MGC-803, KATO-III, and U2OS cells by flow cytometry

In the EMT6-PD-L1 cells, PD-L1 is expressed, while MICA/MICB are not expressed. In the MGC-803 cells, PD-L1 is not expressed, while MICA/MICB are expressed. In the KATO-III cells, PD-L1 is not expressed, and MICA/MICB are also not expressed. In the U2OS cells, PD-L1 is expressed, and MICA/MICB are also expressed.

The EMT6-PD-L1, MGC-803, KATO-III, and U2OS cells in the logarithmic growth phase are taken, centrifuged at 300 g for 5 minutes, counted into cells of 1×10⁶ cells, and washed 1-2 times with the wash buffer. In a 100 μL volume, APC-conjugated anti-human PD-L1 antibody and PE-conjugated anti-human MICA/B antibody are added, incubated on ice in the dark for 30 min, then washed 2-3 times with the wash buffer. Finally, 400 μL of PBS is added to resuspend the cells, and the cells are detected and analyzed by the flow cytometry.

The APC-conjugated anti-human PD-L1 antibody (Cat. No. 2460257) and PE-conjugated anti-human MICA/B antibody (Cat. No. 320906) are purchased from BD Company. The flow cytometer used is BD FACSCanto II.

The PD-L1 detection results are shown in FIG. 4. It can be seen that in the EMT6-PD-L1 cells, the expression level of PD-L1 is 98.1%, while the expression level of MICA/MICB is basically zero; in the MGC-803 cells, the expression level of PD-L1 is basically zero, while the expression level of MICA/MICB is 98.6%; in the KATO-III cells, the expression levels of PD-L1 and MICA/MICB are basically zero; and in the U2OS cells, the expression level of PD-L1 is approximately 84.5% and the expression level of MICA/MICB is approximately 97.6%.

### Embodiment 6

### In Vitro Cytotoxicity Assay of PN-CAR-NK92 Cells

### 1. Cell Preparation

The EMT6-PD-L1, MGC-803, KATO-III and U2OS cells in the logarithmic growth phase after the completion of luciferase transfer are inoculated into a 96-well plate, with 1×10⁴ cells in each well and 3 replicates for each cell, and placed in a 5% carbon dioxide (CO₂) incubator at 37° C. for overnight culture.

### 2. Experimental Design

Two experimental groups are set. In each group, different effector cells are used, including PD1-CAR-NK92, NKG2D-CAR-NK92, PN-CAR-NK92, and NK92 cells, with an effector-to-target ratio of 5:1. Three control groups: target cell control wells, sample maximum enzyme activity control wells, and co-culture wells are set, with a total volume of 100 μL per well. The sample maximum enzyme activity control wells are target cell wells which are not added with effector cell treatment and is used for subsequent lysis.

### 3. Luciferase Detection

In this experiment, ONE-Glo™ Luciferase Assay System Promega is used for detection. Effector cells and target cells are incubated in a 5% CO₂ incubator at 37° C. for 3-6 h. The 96-well plate is taken out from the incubator 1 hour before the predetermined time detection point, and a lysis solution (10×) is added into the sample maximum enzyme activity control well, with an addition amount of 10% of the original culture medium volume. After adding the lysis solution, the mixture is pipetted repeatedly, and then then returned to the incubator for further culture. Once the co-incubation time is reached, a detection reagent is added, and the sample is analyzed using a microplate reader.

Cytotoxicity (%)=(RLUmin-RLUsample)/(RLUmin-RLUmax)×100, where RLU represents a relative luminescence unit.

Results show that for the EMT6-PD-L1 cells, single-module PD1-CAR-NK92 and dual-module PN-CAR-NK92 exhibited significantly higher cytotoxicity than control NK92 group, while NKG2D-CAR-NK92 cells show minimal killing. For the MGC-803 cells, the single-module NKG2D-CAR-NK92 and the dual-module PN-CAR-NK92 show significantly higher cytotoxicity than the control NK92 group, while the PD1-CAR-NK92 cells show minimal killing. For the U2OS cells, at the effector-to-target ratio of 5:1, the PN-CAR-NK92 cells demonstrate 6-fold higher cytotoxicity than the control NK92 group. In contrast, for the KATO-III cells, which do not express PD-L1 or MICA/MICB, no significant change in cytotoxicity is observed.

It should be noted that when claims of the disclosure refer to numerical ranges, it should be understood that the two endpoints of each numerical range and any value between the two endpoints can be selected. To avoid elaboration, the present invention describes illustrated embodiments.

While the illustrated embodiments of the disclosure have been described, those skilled in the art, once apprised of the basic inventive concept, may make additional changes and modifications to these embodiments. Thus, it is intended that the appended claims are intended to be construed as including the illustrated embodiments and all changes and modifications falling within the scope of the disclosure.

It will be apparent to those skilled in the art that various changes and modifications can be made in the disclosure without departing from the spirit and scope of the disclosure. Thus, the disclosure is intended to include such changes and modifications provided they come within the scope of the appended claims and their equivalents.

What is claimed is:

1. A dual-targeting chimeric antigen receptor, comprising a first receptor and a second receptor sequentially connected, each comprising:
a coding sequence of an extracellular recognition and binding domain,
a coding sequence of a transmembrane domain, and
a coding sequence of an intracellular signal transduction domain.

2. The dual-targeting chimeric antigen receptor as claimed in claim 1, wherein:
the extracellular recognition and binding domain of the first receptor is programmed cell death protein 1 (PD1), with the amino acid sequence shown as SEQ ID NO: 3,
the extracellular recognition and binding domain of the second receptor is natural killer group 2, member D (NKG2D), with the amino acid sequence shown as SEQ ID NO: 7; and
the coding sequences of the first receptor and the coding sequences of the second receptor are connected by a cleavage peptide, and the cleavage peptide comprises Foot-and-mouth disease virus 2A (F2A), Thosea asigna virus 2A (T2A), Equine rhinitis virus 2A (E2A), or Porcine teschovirus-1 2A (P2A).

3. The dual-targeting chimeric antigen receptor as claimed in claim 1, wherein:
the extracellular recognition and binding domain is capable of recognizing any tumor-specific or tumor-associated antigen, comprising a PD1 ligands and a NKG2D ligand;
the transmembrane domain and intracellular signal transduction domain are any one or a combination of at least two selected from the group consisting of:
CD28 transmembrane domain and intracellular domain (CD28 TM+ICD),
tumor necrosis factor receptor superfamily member 9 (4-1BB),
DNAX-activating protein of 10 kDa transmembrane domain and intracellular domain (DAP10 TM+ICD),
DNAX-activating protein of 12 kDa (DAP12),
DNAX accessory molecule-1 (DNAM1), and
CD3 zeta chain (CD3ζ).

4. A vector, comprising the dual-targeting chimeric antigen receptor as claimed in claim 1.

5. An immune cell, comprising the dual-targeting chimeric antigen receptor as claimed in claim 1, wherein the immune cell is modified with one or more chimeric molecules.

6. The immune cell as claimed in claim 5, wherein the immune cell is selected from natural killer (NK) cells; and the NK cells comprises: NK cells isolated from human body, stem cell-induced NK cells in vitro, memory NK cells, and NK cell lines.

7. A method for preparing the immune cell as claimed in claim 5, comprising:
introducing the dual-targeting chimeric antigen receptor into immune effector cells.

8. A use of the dual-targeting chimeric antigen receptor as claimed in claim 1, comprising:
preparing a medicament for inhibiting tumor cells using the dual-targeting chimeric antigen receptor or performing tumor cell immunotherapy using the dual-targeting chimeric antigen receptor.

9. A use of the vector as claimed in claim 4, comprising:
preparing a medicament for inhibiting tumor cells using the vector or performing tumor cell immunotherapy using the vector.

10. A use of the immune cell as claimed in claim 5, comprising:
preparing a medicament for inhibiting tumor cells using the immune cell or performing tumor cell immunotherapy using the immune cell.


SEQUENCE LISTING (30 sequences)

SEQ ID NO: 1 - protein, 21 aa, synthetic construct
  REGION 1..21: note = CD8 signal peptide
MALPVTALLLPLALLLHAARP

SEQ ID NO: 2 - protein, 10 aa, synthetic construct
  REGION 1..10: note = Myc tag
EQKLISEEDL

SEQ ID NO: 3 - protein, 147 aa, synthetic construct
  REGION 1..147: note = PD1 extracellular domain
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 4 - protein, 75 aa, synthetic construct
  REGION 1..75: note = DAP10 transmembrane and intracellular domain
QTTPGERSSLPAFYPGTSGSCSGCGSLSLPLLAGLVAADAVASLLIVGAVFLCARPRRSPAQEDGKVYINMPGRG

SEQ ID NO: 5 - protein, 42 aa, synthetic construct
  REGION 1..42: note = CD137 (4-1BB) intracellular domain
KRGRKKLLYIFKQPFMRPVQTTQEEDGCSCRFPEEEEGGCEL

SEQ ID NO: 6 - protein, 8 aa, synthetic construct
  REGION 1..8: note = FLAG tag
DYKDDDDK

SEQ ID NO: 7 - protein, 135 aa, synthetic construct
  REGION 1..135: note = NKG2D ligand-binding domain
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 8 - protein, 12 aa, synthetic construct
  REGION 1..12: note = IgG4 haplotype hinge region
ESKYGPPAPPAP

SEQ ID NO: 9 - protein, 68 aa, synthetic construct
  REGION 1..68: note = CD28 transmembrane and intracellular domain
FWVLVVVGGVLACYSLLVTVAFIIFWVRSKRSRLLHSDYMNMTPRRPGPTRKHYQPYAPPRDFAAYRS

SEQ ID NO: 10 - protein, 42 aa, synthetic construct; identical to SEQ ID NO: 5
  REGION 1..42: note = CD137 (4-1BB) intracellular domain

SEQ ID NO: 11 - protein, 113 aa, synthetic construct
  REGION 1..113: note = CD3 intracellular domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - DNA, 1376 bp, synthetic construct
  misc_feature 1..1376: note = Nucleotide sequence encoding the dual-targeting chimeric antigen receptor
  source 1..1376: mol_type = other DNA
atggcgcttccggtgactgctctacttctaccgttggctctgctgctccacgccgctcgccctgaacagaagctcatctcggaggaagacctgttcctggacagccccgaccgcccttggaatccacccaccttttcaccggccctgctggtggttacagagggcgacaacgccacgttcacctgctccttctccaacacgtcggagagtttcgtcctcaactggtacaggatgtctcccagcaaccagaccgacaaacttgctgcgttccccgaggatcgcagccagccgggccaggattgccgattcagagtcacccagctgcccaacggtcgggactttcatatgtccgtggtccgcgcccgccgcaacgactctggcacctacctgtgcggggccatctctttggctcccaaggcccaaatcaaggagagcctgcgcgccgagctccgcgtaaccgagcggcgtgcagaggtgccaaccgctcacccctctcctagccctaggcctgccggacagttccagaccctggtgcagactactcccggtgagcgcagttccctgcctgcgttttatccggggaccagcggctcctgttctggatgtggttcgctgtccttgcccctgctggccggcttggtggcggcggacgccgtggcttccctgctgattgtcggtgccgtgttcctgtgcgcccggcctcgccgttccccagcgcaggaggacggcaaagtgtacattaatatgcccggccgcggcaagcgcgggcgaaagaagctgctgtacatcttcaagcagccatttatgcgtcctgtgcagacgacccaggaggaagatggctgtagctgccgatttccagaagaagaagaaggaggatgtgaactggaattcgaatttaaaggatcccgcggccgcgctagcggaagcggagctactaacttcagcctgctgaagcaggctggagacgtggaggagaaccctggacctatggccttaccagtgaccgccttgctcctgccgctggccttgctgctccacgccgccaggccggactacaaagacgatgacgacaagttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataat

SEQ ID NO: 13 - protein, 399 aa, synthetic construct
  REGION 1..399: note = Amino acid sequence of the dual-targeting chimeric antigen receptor
MALPVTALLLPLALLLHAARPDYKDDDDKLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPAPPAPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - DNA, 63 bp, synthetic construct
  misc_feature 1..63: note = Nucleotide sequence of the CD8 -C1 signal peptide
  source 1..63: mol_type = other DNA
atggcgcttccggtgactgctctacttctaccgttggctctgctgctccacgccgctcgccct

SEQ ID NO: 15 - DNA, 30 bp, synthetic construct
  misc_feature 1..30: note = Nucleotide sequence of the Myc tag
  source 1..30: mol_type = other DNA
gaacagaagctcatctcggaggaagacctg

SEQ ID NO: 16 - DNA, 441 bp, synthetic construct
  misc_feature 1..441: note = Nucleotide sequence of the PD1 extracellular domain
  source 1..441: mol_type = other DNA
ttcctggacagcccggaccgcccgtggaatccacccaccttttcacccggcctgctggtggttacagagggcgacaacgccacgttcacctgctccttctccaacacgtcggagagtttcgtcctcaactggtacaggatgtctcccagcaaccagaccgacaaacttgctgcgttccccgaggatcgcagccagccgggccaggattgccgattcagagtcacccagctgcccaacggtcgggactttcatatgtccgtggtccgcgcccgccgcaacgactctggcacctacctgtgcggggccatctctttggctcccaaggcccaaatcaaggagagcctgcgcgccgagctccgcgtaaccgagcggcgtgcagaggtgccaaccgctcacccctctcctagccctaggcctgccggacagttccagaccctggtg

SEQ ID NO: 17 - DNA, 224 bp, synthetic construct
  misc_feature 1..224: note = Nucleotide sequence of the DAP10 transmembrane and intracellular domains
  source 1..224: mol_type = other DNA
cagactactcccggtgagcgcagttccctgcctgcgttttatccggggaccagcggctcctgttctggatgtggttcgctgtccttgcccctgctggccggcttggtggcggcggacgccgtggcttccctgctgattgtcggtgccgtgttcctgtgcccccggcctcgccgttcccagcgcaggaggacggcaaagtgtacattaatatgcccggccgcggc

SEQ ID NO: 18 - DNA, 123 bp, synthetic construct
  misc_feature 1..123: note = Nucleotide sequence of the CD137-C1 intracellular domain
  source 1..123: mol_type = other DNA
aagcgcgggcgaaagaagctgctgtacatcttcaagcagccatttatgcgtcctgtgcagacgacccaggaggaagatggctgttcatgcgcttcccggaggaggaggagggcggctgcgagt

SEQ ID NO: 19 - DNA, 24 bp, synthetic construct
  misc_feature 1..24: note = Nucleotide sequence of the FLAG tag
  source 1..24: mol_type = other DNA
gactacaaagacgatgacgacaag

SEQ ID NO: 20 - DNA, 315 bp, synthetic construct
  misc_feature 1..315: note = Nucleotide sequence of the NKG2D ligand-binding domain
  source 1..315: mol_type = other DNA
ttattcaaccaagaagtcaaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaag

SEQ ID NO: 21 - DNA, 35 bp, synthetic construct
  misc_feature 1..35: note = Nucleotide sequence of the IgG4 haplotype hinge region
  source 1..35: mol_type = other DNA
gaatctaaatatgggcctccagcaccaccgcgcct

SEQ ID NO: 22 - DNA, 203 bp, synthetic construct
  misc_feature 1..203: note = Nucleotide sequence of the CD28 transmembrane and intracellular domains
  source 1..203: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctc

SEQ ID NO: 23 - DNA, 123 bp, synthetic construct
  misc_feature 1..123: note = Nucleotide sequence of the CD137-C2 intracellular domain
  source 1..123: mol_type = other DNA
aaacggggcagaaagaaactcctgtatatattcaaacaaccatttatgagaccagtacaaactactcaagaggaagatggctagctgccgatttccagaagaagaagaaggaggatgtgaact

SEQ ID NO: 24 - DNA, 228 bp, synthetic construct
  misc_feature 1..228: note = Nucleotide sequence of the CD3 intracellular domain
  source 1..228: mol_type = other DNA
agagtgaagttcagcaggagcgcagacgcccccgcgtaccagcagggccagaaccagctctataacgagctcaatctaggacgaagagaggagtatgattttctggacaagagacgtggccgggaccctgagatggggggaaagccgcagagaaggaagaaccctcaggaaggcctgtacaatgaactgcagaaagataagatggcggaggcctacagtgagattggg

SEQ ID NO: 25 - DNA, 20 bp, synthetic construct
  misc_feature 1..20: note = PD1-F primer
  source 1..20: mol_type = other DNA
gtgtcacacaactgcccaac

SEQ ID NO: 26 - DNA, 20 bp, synthetic construct
  misc_feature 1..20: note = PD1-R primer
  source 1..20: mol_type = other DNA
ccgcaggctctctttgatct

SEQ ID NO: 27 - DNA, 23 bp, synthetic construct
  misc_feature 1..23: note = NKG2D-F primer
  source 1..23: mol_type = other DNA
gagtgatttttcaacacgatggc

SEQ ID NO: 28 - DNA, 23 bp, synthetic construct
  misc_feature 1..23: note = NKG2D-R primer
  source 1..23: mol_type = other DNA
acagtaactttcggtcaagggaa

SEQ ID NO: 29 - DNA, 20 bp, synthetic construct
  misc_feature 1..20: note = GAPDH-F primer
  source 1..20: mol_type = other DNA
gaggacctgacctgccgtct

SEQ ID NO: 30 - DNA, 20 bp, synthetic construct
  misc_feature 1..20: note = GAPDH-R primer
  source 1..20: mol_type = other DNA
ggaggtgtgggtgtcgctgt